(19) United States
(12) Patent Application Publication
(10) Pub. No.: US 2014/0180171 A1
(43) Pub. Date: Jun. 26, 2014

(54) GAIT-RESPONSIVE ACTIVE TORSO SUPPORT

(71) Applicant: ELWHA LLC, Bellevue, WA (US)

(72) Inventors: Roderick A. Hyde, Redmond, WA (US); Jordin T. Kare, Seattle, WA (US); Dennis J. Rivet, Chesapeake, VA (US); Lowell L. Wood, JR., Bellevue, WA (US)

(73) Assignee: ELWHA LLC, Bellevue, WA (US)

(21) Appl. No.: 13/748,871

(22) Filed: Jan. 24, 2013

Related U.S. Application Data
(63) Continuation-in-part of application No. 13/721,474, filed on Dec. 20, 2012.
Application No. 13/721,474 is a continuation-in-part of application No. 13/739,868, filed on Jan. 11, 2013.

(51) Int. Cl. A61B 5/11

(52) U.S. Cl. CPC A61B 5/112
USPC 600/595

(57) ABSTRACT

An active torso support that controls force applied to one or more portions of a torso of a subject in response to detected gait of the subject is described. For example, the torso support can be a back brace for providing support to the back of a subject to limit or prevent injury or discomfort. The active torso support includes one or more elements for applying force to the torso of the subject, positioned on the torso of the subject by a positioning element, which may include a belt, for example. Gait of the subject can be determined through analysis of signals from one or more sensors located on the active torso support and/or at a location remote from the active torso support.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### Priority Applications:

For purposes of the USPTO extra-statutory requirements, the present application constitutes a continuation-in-part of U.S. patent application Ser. No. 13/721,474, entitled POSTURE DEPENDENT ACTIVE TORSO SUPPORT, naming RODERICK A. HYDE, JORDIN T. KARE, DENNIS J. RIVET, AND LOWELL L. WOOD, JR. as inventors, filed 20 Dec. 2012 with attorney docket no. 1108-004-001-000000, which is currently co-pending or is an application of which a currently co-pending application is entitled to the benefit of the filing date.
For purposes of the USPTO extra-statutory requirements, the present application constitutes a continuation-in-part of U.S. patent application Ser. No. 13/739,868, entitled POSITION SENSING ACTIVE TORSO SUPPORT, naming RODERICK A. HYDE, JORDIN T. KARE, DENNIS J. RIVET, AND LOWELL L. WOOD, JR. as inventors, filed 11 Jan. 2013 with attorney docket no. 1108-004-012-000000, which is currently co-pending or is an application of which a currently co-pending application is entitled to the benefit of the filing date.

### Related Applications:

None.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

To the extent that the listings of applications provided above may be inconsistent with the listings provided via an ADS, it is the intent of the Application to claim priority to all applications listed in the Priority Applications section of either document.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In one aspect, an active torso support includes, but is not limited to, at least one force applying element adapted to apply force to a portion of a torso of a subject; at least one positioning element adapted to position the at least one force applying element with respect to the torso of the subject; and control circuitry including: gait analysis circuitry configured to determine a gait of the subject responsive to a signal containing information indicative of the gait of the subject; and actuation circuitry configured to control actuation of the at least one force applying element responsive to the signal indicative of the gait of the subject. In an aspect the active torso support includes at least one sensor on the torso support adapted to produce the signal containing information indicative of the gait of the subject. In an aspect, the active torso support is operably coupled to a remote device including at least one sensor adapted to produce the signal containing information indicative of the gait of the subject. In addition to the foregoing, other system aspects are described in the claims, drawings, and text forming a part of the disclosure set forth herein.

In one aspect, a method of controlling an active torso support includes, but is not limited to, receiving a gait signal containing information indicative of a gait of a subject wearing an active torso support, wherein the active torso support includes at least one force applying element adapted to apply force to a portion of a torso of the subject and at least one positioning element adapted to position the at least one force applying element with respect to the torso of the subject; determining the gait of the subject based at least in part on the received signal; and controlling actuation of at least one force applying element to apply force to the portion of the torso of the subject based on the gait of the subject. In addition to the foregoing, other method aspects are described in the claims, drawings, and text forming a part of the disclosure set forth herein.

In one aspect, an article of manufacture includes, but is not limited to, one or more non-transitory machine-readable data storage media bearing one or more instructions for: receiving a gait signal containing information indicative of a gait of a subject wearing an active torso support, wherein the active torso support includes at least one force applying element adapted to apply force to a portion of a torso of the subject and at least one positioning element adapted to position the at least one force applying element with respect to the torso of the subject; determining the gait of the subject based at least in part on the received signal; and controlling actuation of at least one force applying element to apply force to the portion of the torso of the subject based on the gait of the subject. In addition to the foregoing, other aspects of articles of manufacture including one or more non-transitory machine readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

For a more complete understanding of embodiments, reference now is made to the following descriptions taken in connection with the accompanying drawings. The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 is an illustration of a torso support.

FIG. 2 is an illustration of a torso support in use.

FIG. 3 is a block diagram of a torso support.

FIG. 4 is a flow diagram of a method of controlling a torso support.

FIG. 5 is a flow diagram of a method of controlling a torso support.

FIG. 6 is a flow diagram of a method of controlling a torso support.

FIG. 7 is a flow diagram of a method of controlling a torso support.

FIG. 8 illustrates an article of manufacture including non-transitory machine-readable data storage media bearing one or more instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

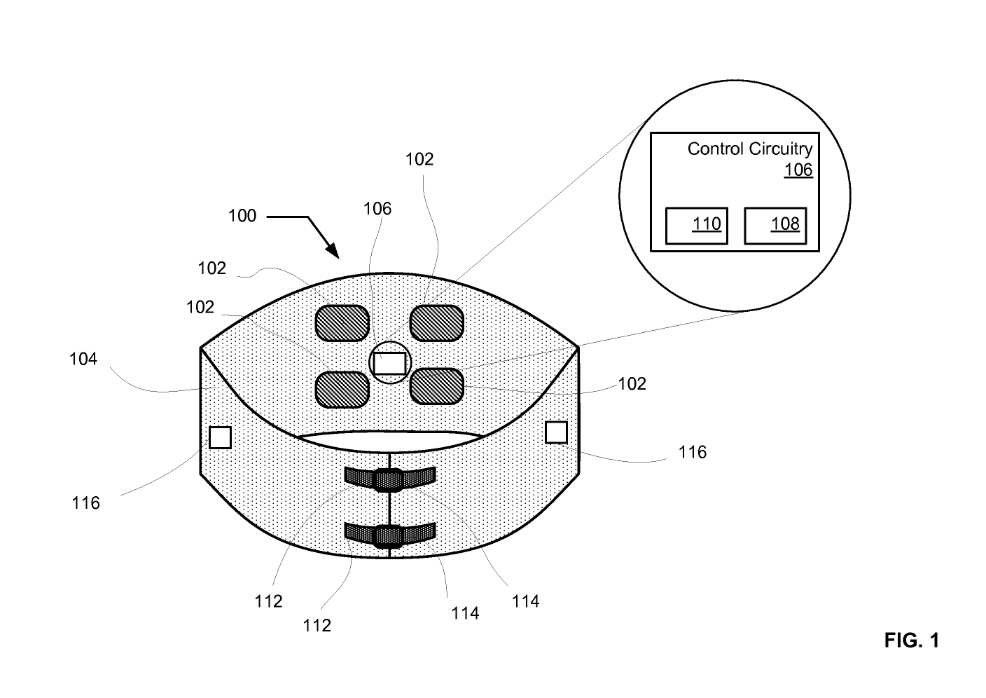

FIG. 1 depicts an active torso support 100 that includes at least one force applying element 102 adapted to apply force to a portion of a torso of a subject; at least one positioning element 104 adapted to position the at least one force applying element with respect to the torso of the subject; and control circuitry 106 including: gait analysis circuitry 108 configured to determine a gait of the subject responsive to a signal containing information indicative of the gait of the subject; and actuation circuitry 110 configured to control actuation of the at least one force applying element responsive to the signal indicative of the gait of the subject.

Active torso support 100 may include one or multiple force applying elements 102 that are capable of applying force or pressure to a region of the torso of the subject, for example, for the purpose of providing support to weak or injured muscles and/or to prevent or minimize discomfort or injury to muscles or other structures in the torso due to loading. The active torso support may be configured as a back support or back brace, as depicted in FIG. 1, but is not limited thereto, and may be configured to support or brace other portions of the torso, including, for example, portions of a back, a side, an abdomen, a chest, a ribcage, a stomach, a hip, a pelvic region, a thoracic region, a shoulder region, a pectoral region, a buttock, a lower back, or an upper back.

It is contemplated that an active torso support as described herein functions generally as follows: if a particular gait or change in gait of the subject is known to produce motion or loading of muscles and/or bony structures in the subject's torso that is likely to result in injury or discomfort, the active torso support will respond to detection of that gait or change in gait by applying force to one or more appropriate portions of the torso to provide support expected to prevent or minimize injury or discomfort. In an aspect, the active torso support may respond to detection of gait or change in gait by reducing the amount of force applied to one or more portions of the torso, e.g. to permit greater freedom of movement.

A force applying element (e.g. force applying element 102 depicted in FIG. 1) can be any structure that is capable of applying force to a region of the torso of the subject, via a torso-contacting portion such as a pad or probe, and a controllable force-generating component that acts to move the torso contacting portion relative to the torso (e.g. by pressing against the torso and/or by applying shear forces to the torso, e.g. by engaging the surface of the torso by friction). A controllable force generating component can be controlled by control circuitry 106, e.g. via an electrical signal carried via an electrical connection or via a wireless signal such as an optical or electromagnetic signal transmitted from the control circuitry to the force applying element. Force applying element 102 may include one or more actuator, mechanical linkage, expandable element, inflatable element, pneumatic element, or hydraulic element, or other structures or components capable of applying force or pressure in a controlled fashion to a localized area of the torso. A force applying element can be adapted to fit against a portion of the torso of the subject, where the portion of the torso of the subject is selected from a back, a side, an abdomen, a chest, a ribcage, a stomach, a hip, a pelvic region, a thoracic region, a shoulder region, a pectoral region, a buttock, a lower back, and an upper back. Size, configuration, and force-applying capability of the force applying element are adapted for use with the selected portion of the torso.

A force applying element may be adapted to apply force to the torso of the subject with at least a component of the force in a direction normal to the surface of the torso of the subject. For example, a force applying element can include a plate (which may be curved or planar) a probe, or any structure having shape and size suitable for applying force to a desired portion of the torso. A force applying element can also include a skin-engaging element adapted to apply tensile or shear force to the skin surface; for example a skin-engaging element may include an adhesive, suction cup, or a frictional surface, or other components known to those skilled in the art to provide for the application of tensile or shear forces to the skin. Thus, a force applying element can be adapted to apply force to the torso of the subject with at least a component of the force in a direction tangential to the surface of the torso of the subject. In an aspect, the force applying element includes a passive force applying element and a controllable active force applying element. In an aspect, the force applying element has a controllable stiffness, a controllable dimension, and/or a controllable position relative to the positioning element. The force applying element can include one or more of a spring, an elastic material, or a viscoelastic material. In an aspect, the force applying element includes an actuator, which may include, for example, a mechanical linkage, an expandable element, an inflatable element, a screw, a pneumatic element, or a hydraulic element.

Expandable fluid/air filled bladders, are described, for example, in U.S. Pat. No. 4,135,503 to Romano; U.S. Pat. No. 6,540,707 to Stark et al, and U.S. Pat. No. 5,827,209 to Gross et al., each of which is incorporated herein by reference. Expansion of such bladders can be controlled through the use of a motorized pump and electrically controlled valves, with feedback provided by pressure sensors. Mechanically or pneumatically driven force applying elements can be, e.g. as described in U.S. Pat. No. 5,624,383 to Hazard et al., which is incorporated herein by reference. Pneumatic and hydraulic piston type force applying elements as described in U.S. Pat. No. 6,746,413 to Reinecke et al., which is incorporated herein by reference, and screw thread/worm gear assembly structures as described in U.S. Published Patent Application 2009/0030359 to Wikenheiser et al., which is incorporated herein by reference, may be positioned to press against the torso (delivering force substantially perpendicular to the skin surface), or positioned to apply shear forces (i.e., force having a significant component parallel to the skin surface).

Although positioning element 104 is depicted in FIG. 1 as a belt adapted to be fitted around the waist/mid-torso of a subject, the positioning element can be any structure capable of holding force applying elements 102 in position with regard to at least a portion of the torso of the subject, and may include, for example, at least one band, strap, belt, harness, or a garment such as a corset, girdle, jacket, vest, or brief. The positioning element may include one or multiple straps or other components, without limitation. The positioning element can be constructed from flexible, resilient, or elastic material, including but not limited to leather, fabric, webbing, mesh, cable, cord, flexible metals or polymers, or sections of rigid metals, polymers or other materials connected in such a manner that the sections can be movably fitted around the torso of the subject, e.g. by a hinge or other linkage or by one or more sections of flexible material. Positioning element 104 may include fasteners to secure the positioning element with respect to the torso of the subject, e.g. straps 112 and buckles 114 as depicted in FIG. 1, or other fasteners as are known in the art, including but not limited to buckles, snaps, zippers, latches, clips, ties, hook and loop fasteners, lacings, and so forth. Positioning element may include an active or passive tensioning component (for example, elastic) to provide for tightening of the positioning element about the torso of the subject to provide for a secure fit. In an embodiment, positioning element may simply include an elastic component which allows it to be slid onto the torso of the subject, without the need for fasteners.

Force applying elements 102, control circuitry 106, and other system components described herein may be attached to the positioning element 104 or held in place by pressure or friction, e.g. by being pressed between the torso of the subject and the positioning element.

In an aspect, active torso support 100 includes at least one sensor 116 carried by positioning element 104 on the torso support adapted to produce the signal containing information indicative of the gait of the subject. For example, sensor 116 is mounted directly on positioning element 104 or mounted on or otherwise attached to another component carried by positioning element 104. For example, FIG. 1 depicts active torso support 100 including two sensors 116, which include accelerometers, for example, a tri-axial accelerometer or an integrating accelerometer. Detection of gait based on signals from accelerometers is performed, for example, as described by Derawi et al., “Improved Cycle Detection for Accelerometer Based Gait Authentication,” IEEE Sixth International Conference on Intelligent Information Hiding and Multimedia Signal Processing,” Oct. 15-17, 2010, pp. 312-317; Sabelman et al., “Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784; Rong et al., “A Wearable Acceleration Sensor System for Gait Recognition,” 2007 Second IEEE Conference on Industrial Electronics and Applications, May 23-25, 2007, pp. 2654-2659; and Sekine et al., “Discrimination of Walking Patterns Using Wavelet-Based Fractal Analysis,” IEEE Transactions on Neural Systems and Rehabilitation Engineering, Vol. 10, No. 3, September 2002, pp. 188-196, each of which is incorporated herein by reference. The torso support can include other types of sensors, including but not limited to gyro sensors (e.g., to indicate inclination or leaning over of the subject), magnetometers (which provide angle information, or can be used with external field coils to provide both position and angle), and differential position sensors (using GPS or pseudo-GPS signals). A torso support can include one or multiple sensors, without limitation.

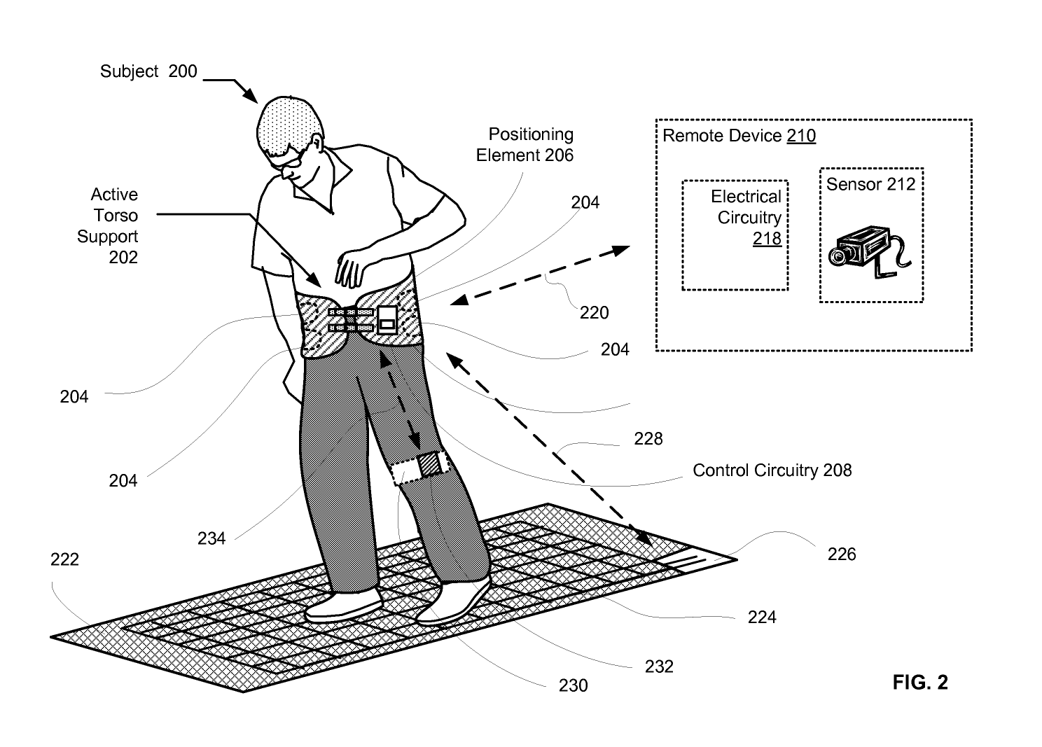

FIG. 2 depicts a subject 200 wearing an active torso support 202, which includes force applying elements 204, positioning element 206, and control circuitry 208. In an aspect, active torso support 202 is operably coupled to a remote device 210 that includes at least one sensor 212 adapted to produce the signal containing information indicative of the gait of the subject. Remote device 210 is thus a functional component of active torso support 202, in that it is operably coupled to other components of active torso support 202. Remote device 210 can include a sensor 212 that includes a camera system, as depicted in FIG. 2. The remote device 210 may be located in the environment of the subject. For example, the camera (sensor 212) in FIG. 2 is part of remote device 210 mounted in the environment of the subject, which includes an area occupied by the subject, for example a bedroom, an office, a vehicle, a hospital room, a room of a care facility, etc. Electrical circuitry 218 provides for data processing and transmission of a data signal 220 to active torso support 202. Detection of gait based on image analysis can be performed, by various methods, e.g. as described in U.S. Pat. No. 7,330,566, issued Feb. 12, 2008 to Cutler, or U.S. Pat. No. 7,728,839 issued Jun. 1, 2010 to Yang et al., each of which is incorporated herein by reference.

Remote devices suitable for use in connection with active torso support 202 can include other types of sensors, and can be located in the environment (see for example, remote device 222) or located on the body of the subject at a position remote from the active torso support (see, for example, remote device 230). In an aspect, an active torso support 202 can receive information from multiple remote devices that include sensors. A remote device in the environment of the subject can include a sensor on stairs or a floor. For example, FIG. 2 depicts remote device 222 configured as a mat including resistive sensor grid 224 (as described in Middleton et al., “A floor sensor system for gait recognition,” Fourth IEEE Workshop on Automatic Identification Advanced Technologies, 2005, pp. 171-176, Digital Object Identifier: 10.1109/AUTOID.2005.2, which is incorporated herein by reference), and electrical circuitry 226, configured to transmit data signal 228 containing information regarding gait parameters such as stride length and stride cadence to control circuitry 208 on active torso support 202.

In another aspect, the active torso support 202 is operably coupled to a remote 230 device located on the body of the subject and including at least one sensor 232. Sensor 232 can include, but is not limited to a gyro (e.g., to indicate inclination or leaning over of the subject), force sensor, pressure sensor, accelerometer (which may be a tri-axial accelerometer or an integrating accelerometer), magnetometer (which can be used to provide angle information, or can be used with external field coils to provide both position and angle), or differential position sensors (using GPS or pseudo-GPS signals). For example, accelerometers located on various portions of the body can be used to provide signals indicative of the gait of the subject, including on the legs (see, e.g. Torrealba et al., “Statistics-based technique for automated detection of gait events from accelerometer signals,” Electronics Letters, 28 Oct. 2010, Vol. 46, No. 22, and Itoh et al., “Development of New Instrument for Evaluating Leg Motions Using Acceleration Sensors,” Environmental Health and Preventive Medicine 12, 111-118, May 2007, each of which is incorporated herein by reference), legs and/or arms (see Mannini et al., “Accelerometry-Based Classification of Human Activities Using Markov Modeling,” Computational Intelligence and Neuroscience, Vol. 2011, Article ID 647858, published online 4 Sep. 2011, which is incorporated herein by reference), and/or head (see Sabelman et al., “Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784, which is incorporated herein by reference). A data signal 234 is sent from remote device 230 to control circuitry 208, as described generally herein above. Remote devices, such as remote devices 210, 222, and 230, are functional components of active torso support 202, in that they are operably coupled to other components of active torso support 202.

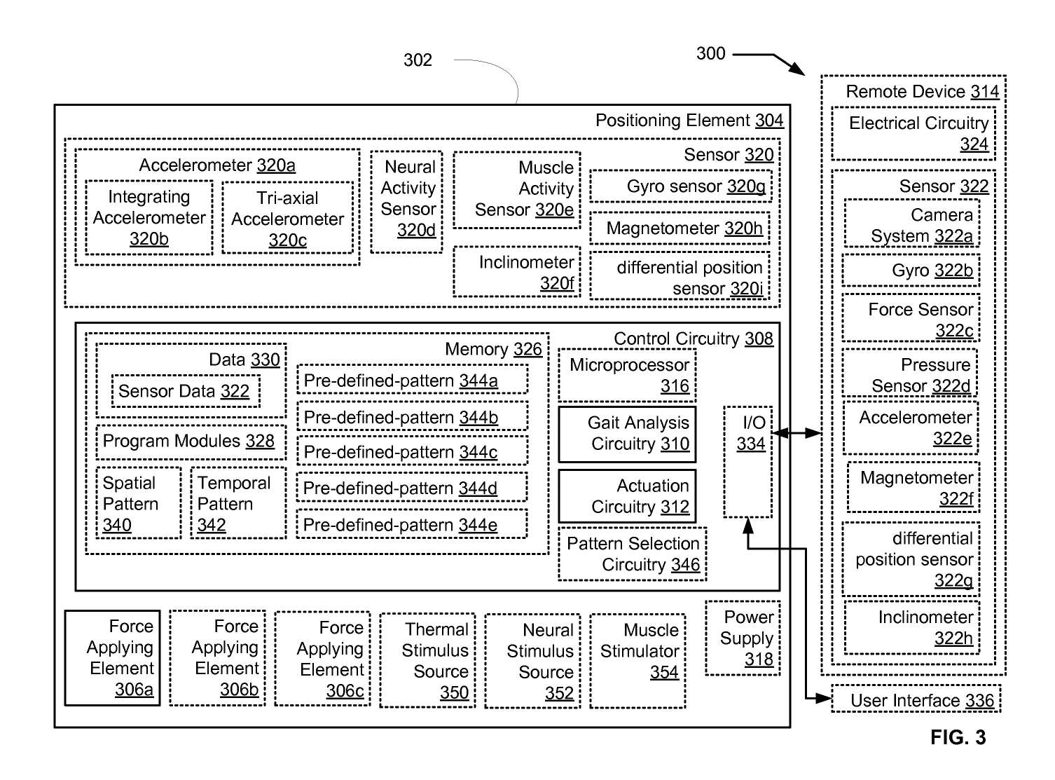

FIG. 3 is a block diagram depicting components of an active torso support system 300, including active torso support 302 including positioning element 304 and one or more force applying elements 306a-306c, and control circuitry 308 including gait analysis circuitry 310 and actuation circuitry 312. Gait analysis circuitry 310 is configured to determine a gait of the subject responsive to a signal containing information indicative of the gait of the subject and actuation circuitry 312 is configured to control actuation of the at least one force applying element responsive to the signal indicative of the gait of the subject. Three force applying elements 306a-306c are depicted in FIG. 3, for the purpose of illustration. However, in some embodiments, only a single force applying element may be used, while in other embodiments, larger numbers of force applying elements may be used. Force applying elements are as described in connection with FIG. 1, and are typically electromechanical in nature. It will be appreciated that a wide range of components may impart mechanical force or motion, such as rigid bodies, spring or torsional bodies, hydraulics, electro-magnetically actuated devices, and/or virtually any combination thereof. As used herein “electro-mechanical system” includes, but is not limited to, electrical circuitry operably coupled with a transducer (e.g., an actuator, a motor, a piezoelectric crystal, a Micro Electro Mechanical System (MEMS), etc). Those skilled in the art will recognize that electro-mechanical as used herein is not necessarily limited to a system that has both electrical and mechanical actuation except as context may dictate otherwise.

In some embodiments, torso support system 300 includes a remote device 314, e.g. as in the example depicted in FIG. 2. Control circuitry 308 may include analog or digital circuitry electrical circuitry. In an aspect, control circuitry 308 includes a microprocessor 316. Active torso support 302 may include various other components, including power supply 318 and one or more sensors 320 (e.g. sensors 320a-320i). A sensor 320 can include an accelerometer 320a, for example, which may be an integrating accelerometer 320b or a tri-axial accelerometer 320c. Alternatively, or in addition, remote device 314 may include one or more sensors 322, as well as electrical circuitry 324. Sensors 322 in remote device 314 can include a camera system 322a, gyro 322b, force sensor 322c, pressure sensor 322d, accelerometer 322e (e.g., a tri-axial accelerometer or an integrating accelerometer), magnetometer 322f, differential position sensor 322g, or inclinometer 322h, as discussed herein above in connection with FIG. 2. Control circuitry 308 may include memory 326, which may store program modules 328 used in the operation of active torso support system 300, and/or data 330, which may include, for example, sensor data 332 from one or more sensors 320 or 322. Control circuitry 308 may include I/O structure 334, which provides for communication with remote device 314, e.g. via a wired or wireless (e.g. electromagnetic or optical) connection, or with a user interface 336. Electrical circuitry 324 in remote device 314 includes any electrical circuitry needed for processing signal from sensors 322 and sending signals to or receiving signals from active torso support 302 via I/O structure 334.

In an aspect, torso support 302 includes a neural activity sensor 320d adapted to sense neural activity. In another aspect, torso support 302 includes a muscle activity sensor 320e adapted to sense muscle activity. An electromagnetic sensor (e.g. a surface electrode) may be used for sensing electrical activity produced by a nerve, nerve plexus, or other neural structure, or by a muscle (including cardiac or skeletal muscle) below the skin, as described for example in U.S. Pat. No. 8,170,656 issued May 1, 2012, to Tan et al., which is incorporated herein by reference. Magnetic fields produced by neural activity can be sensed, for example, by a magnetometer, e.g. as described by Sander et al. in “Magnetoencephalography with a chip-scale atomic magnetometer,” Biomedical Optics Express, May 2012, Vol. 3, No. 5, p. 982, which is incorporated herein by reference. Sensed neural activity may provide information about the gait of the subject, or about pain or other sensations of the subject. Sensed muscle activity may provide information about the gait or muscle fatigue, for example.

Actuation circuitry 312 is configured to control actuation of the at least one force applying element (e.g. 306a-306c) responsive to a change in the gait of the subject. For example, actuation circuitry 312 can be configured to control actuation of the at least one force applying element 306a-306c as a function of the speed of the gait. In an aspect, actuation circuitry 312 is configured to control actuation of the at least one force applying element 306a-306c to provide additional support to the torso of the subject responsive to detection of the change in the gait of the subject. In another aspect, actuation circuitry 312 is configured to control actuation of the at least one force applying element 306a-306c to provide less support to the torso of the subject responsive to detection of the change in the gait of the subject.

Gait analysis circuitry 310 may be configured to detect a change in the gait of the subject to walking, running, climbing stairs, or descending stairs, or from these or other active gaits to standing still. For example, Mannini et al. describe processing of signals from accelerometers worn on a subject's hip, wrist, arm, ankle and thigh to distinguish a variety of activities, including walking, running, standing, and climbing stairs (see Mannini et al., “Accelerometry-Based Classification of Human Activities Using Markov Modeling,” Computational Intelligence and Neuroscience, Vol. 2011, Article ID 647858, published online 4 Sep. 2011, and Sekine et al., “Discrimination of Walking Patterns Using Wavelet-Based Fractal Analysis,” IEEE Transactions on Neural Systems and Rehabilitation Engineering, Vol. 10, No. 3, September 2002, pp. 188-196, each of which is incorporated herein by reference.) Gait analysis circuitry 310 may be configured to detect a change in the gait of the subject indicative of the subject stumbling, subject falling, or changing direction. Data from accelerometers located on the hips of a subject can be used to distinguish walking, turning, ascending or descending stairs, as described in Sabelman et al., (“Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784), which is incorporated herein by reference.

In an aspect, active torso support 302 includes an inclinometer 320f adapted to generate an inclination signal indicative of an inclination of at least a portion of the active torso support. Inclinometer 320f can be, but is not limited to, a MEMS type digital inclinometer (for example, an Analog Devices ADIS 16209) that can be used to detect the inclination of the subject's torso. Examples of other suitable sensors are gyro sensor 320g, magnetometer 320h, and differential position sensor 320i. An inclinometer can be used in combination with other sensors to provide information regarding the angular position of the subject's limbs or spine, which is indicative of aspects of the subjects gait, and may also provide information regarding disturbances in gait, including tilting, swaying or falling. In connection therewith, gait analysis circuitry 310 may be configured to generate a signal indicative of the gait of the subject based at least in part on the inclination signal. Similarly, actuation circuitry 312 may be configured to control actuation of the at least one force applying element 306a-306c responsive to the signal indicative of the gait of the subject based at least in part on the inclination signal.

In an aspect, actuation circuitry 312 is configured to control actuation of the at least one force applying element 306a-306c according to a temporal pattern 342 based at least in part on the signal indicative of the gait of the subject. Controlling actuation according to a temporal pattern may be as simple as applying a constant force at a selected location for a specific duration (e.g., a duration corresponding to an expected duration of a particular motion, such as a portion of a gait cycle), or applying a force that gradually ramps up to a maximum value as a function of time.

In some embodiments, active torso support 302 includes at least two force applying elements 306a-306c adapted to apply force to at least two different portions of the torso of the subject, wherein the actuation circuitry is configured to control actuation of the at least two force applying elements 306a-306c according to a spatial pattern 340 and/or temporal pattern 342 based at least in part on the signal indicative of the gait of the subject. For example, a spatial pattern provides for applying force at several spatially separated locations to support several different muscles (or different portions of a larger muscle) that are loaded or stressed during a particular gait. More complex temporal or spatio-temporal patterns (e.g. cyclical patterns) may also be employed. Cyclical patterns may be matched to the gait cycle, for example.

In an aspect, active torso support 302 includes a memory 326 adapted to store a plurality of pre-defined patterns 344a-344e (again, the specific number of pre-defined patterns depicted in FIG. 3 is for the sake of illustration only, and larger or smaller numbers of patterns may be used in other aspects), wherein the actuation circuitry 312 is configured to control actuation of the at least one force applying element 306a-306c according to a pre-defined pattern selectable from the plurality of pre-defined patterns 344a-344e.

In an aspect, active torso support 302 includes a user input, wherein the pre-defined pattern is selectable from the plurality of pre-defined patterns by a user via the user input. The user input may include, for example, user interface 336. Active torso support 302 may also include pattern selection circuitry 346 configured to select the pre-defined pattern from the plurality of pre-defined patterns 344a-344e based at least in part upon the gait of the subject.

Force applying elements (e.g. 306a-306c) may include, for example, at least one actuator, mechanical linkage, expandable element, inflatable element, pneumatic element, or hydraulic element, as discussed herein above.

In an aspect, actuation circuitry 312 is configured to control a pattern of force generated by the at least one force generating element. In another aspect, control circuitry is adapted to control a pattern of motion generated by the at least one force generating element.

In an aspect, active torso support 302 includes thermal stimulus source 350 configured to deliver a thermal stimulus to at least a portion of the torso of the subject. Thermal stimulus source 350 may include, for example, a resistive element, an infrared source, a microwave source, an acoustic energy source, or other elements capable of providing localized heating to the skin or underlying tissues. A thermal stimulus may be applied to stimulate blood circulation, promote healing, enhance comfort of sore or injured muscles, or serve as a counter-stimulus to reduce sensation of pain, for example.

In an aspect, active torso support 302 includes neural stimulus source 352 configured to deliver a stimulus to a neural structure in the torso of the subject. In an aspect, active torso support 302 includes a muscle stimulator 354 configured to deliver a stimulus to a muscle in the torso of the subject. A neural stimulator 352 or muscle stimulator 354 may include an electrode for delivering an electrical stimulus, or one or more coils for delivering a magnetic stimulus, for example, either of which can be driven by an appropriately configured electrical control signal, as known to those having skill in the art. (See, for example, U.S. Pat. No. 8,285,381 issued Oct. 9, 2012 to Fahey et al., which is incorporated herein by reference). Other types of neural or muscle stimulators may be used, as known to those having skill in the art. Nerve and/or muscle stimulation can be used to activate muscles to provide a higher level of strength or stability in the back, or to block or counter pain signals, for example.

In a general sense, those skilled in the art will recognize that the various embodiments described herein can be implemented, individually and/or collectively, by various types of electrical circuitry having a wide range of electrical components such as hardware, software, firmware, and/or virtually any combination thereof, limited to patentable subject matter under 35 U.S.C. §101. Electrical circuitry (including control circuitry 308 and electrical circuitry 324 depicted in FIG. 3) includes electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc), and/or any non-electrical analog thereto, such as optical or other analogs (e.g., graphene based circuitry). In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

Those skilled in the art will recognize that at least a portion of the devices and/or processes described herein can be integrated into a data processing system. Those having skill in the art will recognize that a data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

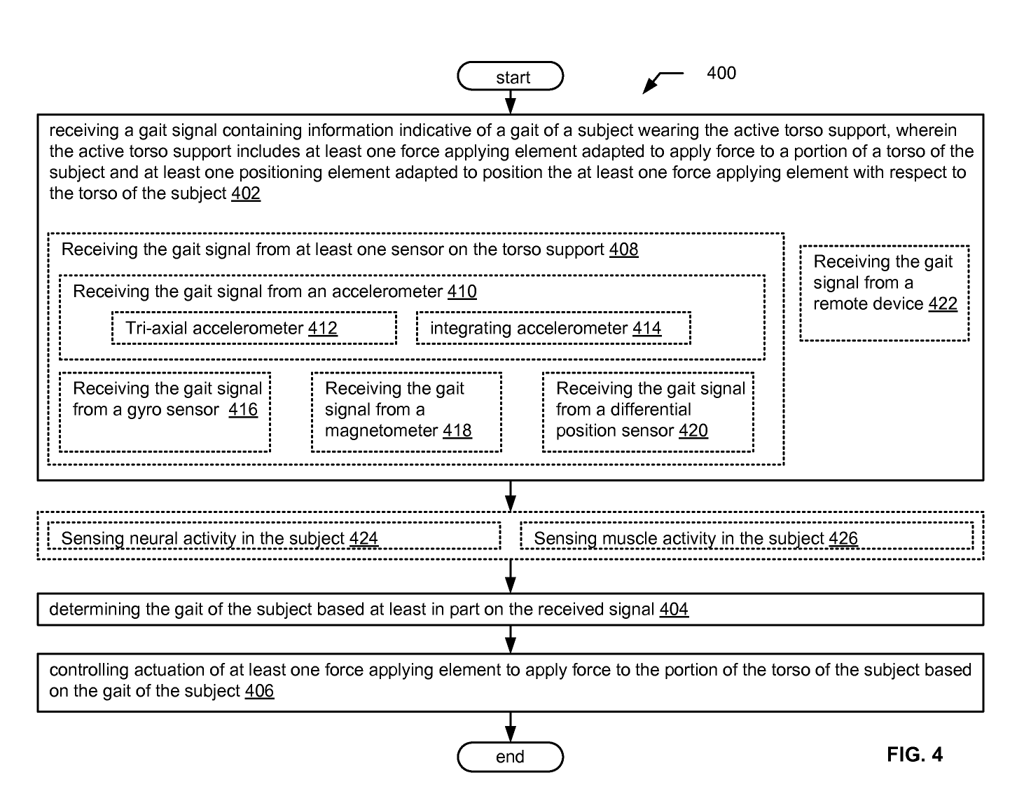

FIG. 4 is a flow diagram of a method of controlling an active torso support, including receiving a gait signal containing information indicative of a gait of a subject wearing an active torso support, wherein the active torso support includes at least one force applying element adapted to apply force to a portion of a torso of the subject and at least one positioning element adapted to position the at least one force applying element with respect to the torso of the subject at 402; determining the gait of the subject based at least in part on the received signal at 404; and controlling actuation of at least one force applying element to apply force to the portion of the torso of the subject based on the gait of the subject at 406.

In aspect, method 400 includes receiving the gait signal from at least one sensor on the torso support at 408, which may be, for example, an accelerometer 410 (e.g., tri-axial accelerometer 412 or integrating accelerometer 414), a gyro sensor 416, magnetometer 418, or differential position sensor 420. In an aspect, method 400 includes receiving the gait signal from a remote device, at 422. Method 400 may include sensing neural activity in the subject, as indicated at 424 or sensing muscle activity in the subject, as indicated at 426.

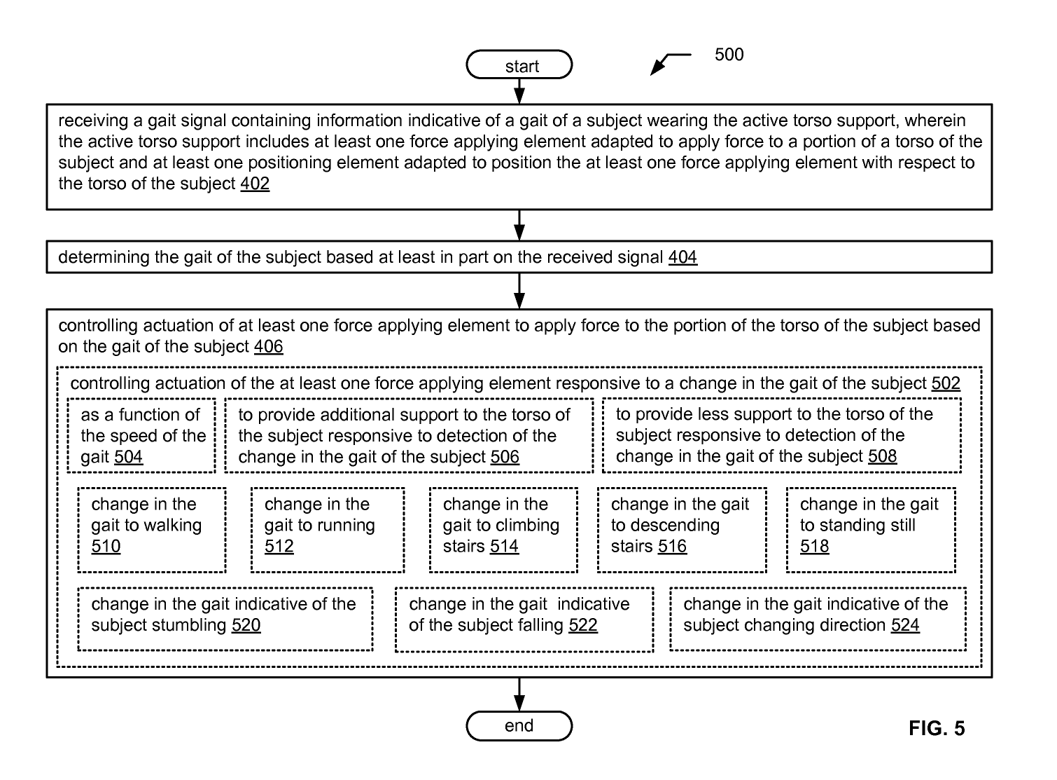

Variants of the basic method depicted in FIG. 4 are shown in FIGS. 5-7. In these figures, steps 402, 404, and 406 are as described in connection with FIG. 4. In an aspect, a related method 500, as shown in FIG. 5, includes controlling actuation of the at least one force applying element responsive to a change in the gait of the subject, as indicated at 502, for example, by controlling actuation of the at least one force applying element as a function of the speed of the gait at 504, or controlling actuation of the at least one force applying element to provide additional support to the torso of the subject responsive to detection of the change in the gait of the subject at 506, or to provide less support to the torso of the subject responsive to detection of the change in the gait of the subject, at 508. For example, method 500 may include controlling actuation of the at least one force applying element responsive to change in the gait of the subject to walking 510, running 512, climbing stairs 514, descending stairs 516, or to standing still 518 from these or other active gaits. The method can include controlling actuation of the at least one force applying element responsive to change in the gait of the subject indicative of the subject stumbling 520, falling 522, or changing direction 524.

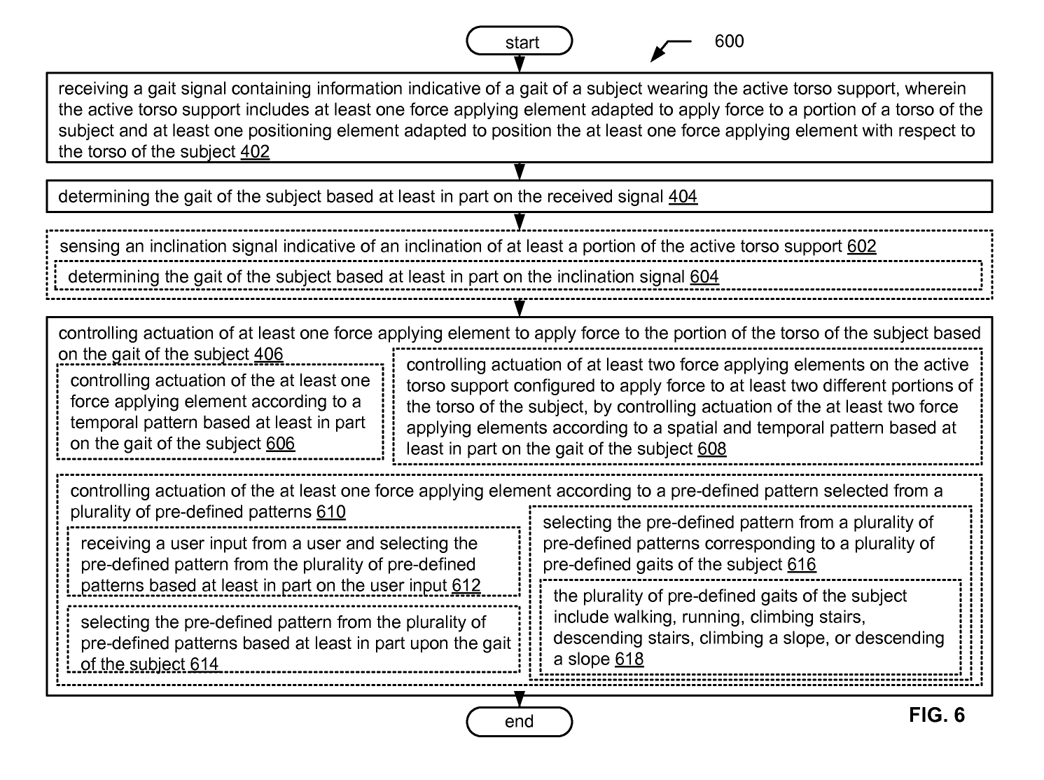

As depicted in FIG. 6, in an aspect, a method 600 includes sensing an inclination signal indicative of an inclination of at least a portion of the active torso support 602. Furthermore, method can include determining the gait of the subject based at least in part on the inclination signal at 604.

In an aspect, method 600 includes controlling actuation of the at least one force applying element according to a temporal pattern based at least in part on the gait of the subject 606. In an aspect, method 600 includes controlling actuation of at least two force applying elements on the active torso support configured to apply force to at least two different portions of the torso of the subject, by controlling actuation of the at least two force applying elements according to a spatial and temporal pattern based at least in part on the gait of the subject, at 608.

In another aspect, method 600 includes controlling actuation of the at least one force applying element according to a pre-defined pattern selected from a plurality of pre-defined patterns 610. The method may include receiving a user input from a user and selecting the pre-defined pattern from the plurality of pre-defined patterns based at least in part on the user input 612. In an aspect, the method includes selecting the pre-defined pattern from the plurality of pre-defined patterns based at least in part upon the gait of the subject 614. In an aspect, the method includes selecting the pre-defined pattern from a plurality of pre-defined patterns corresponding to a plurality of pre-defined gaits of the subject 616, which may be, for example walking, running, climbing stairs, descending stairs, climbing a slope, or descending a slope as indicated at 618. Additionally, one of the plurality of pre-defined patterns may correspond to standing still.

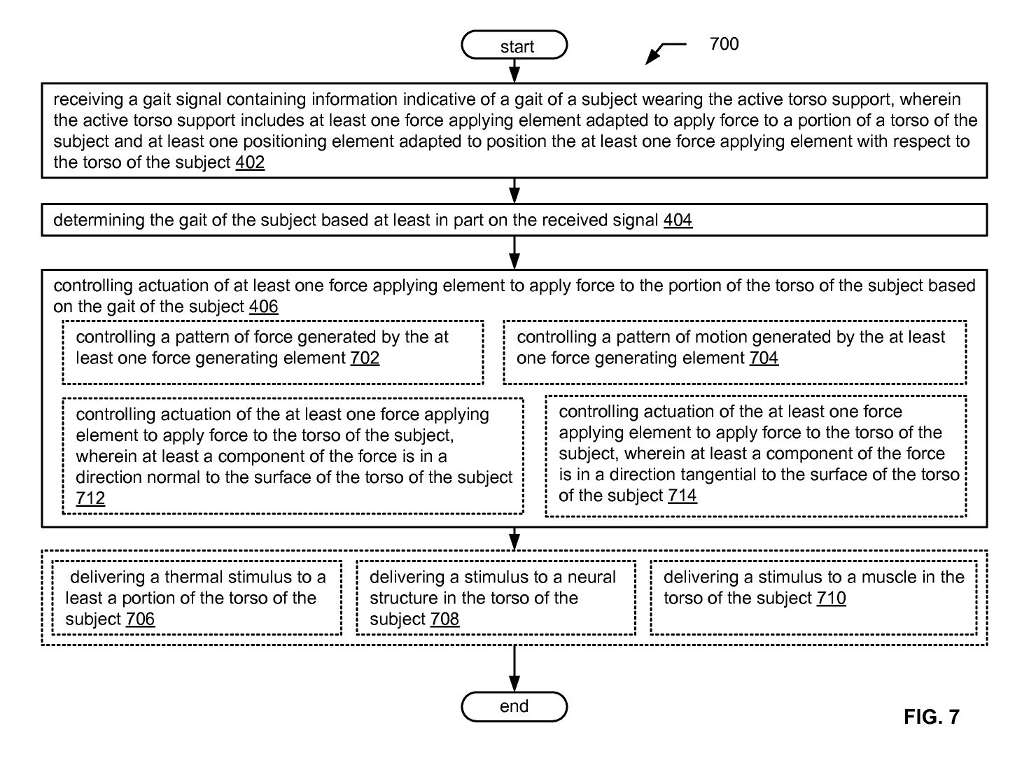

Additional method aspects are shown in FIG. 7. For example, in method 700, controlling actuation of the at least one force applying element can include controlling a pattern of force generated by the at least one force generating element as indicated at 702, or controlling a pattern of motion generated by the at least one force generating element, as indicated at 704.

Method 700 can include delivering a thermal stimulus to a least a portion of the torso of the subject 706, delivering a stimulus to a neural structure in the torso of the subject 708, or delivering a stimulus to a muscle in the torso of the subject 710.

In an aspect, method 700 includes controlling actuation of the at least one force applying element to apply force to the torso of the subject, wherein at least a component of the force is in a direction normal to the surface of the torso of the subject 712. As discussed herein above, a force normal to the surface of the torso can be a compressive force or a tensile force. In an aspect, method 700 includes controlling actuation of the at least one force applying element to apply force to the torso of the subject, wherein at least a component of the force is in a direction tangential to the surface of the torso of the subject 714.

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture, limited to patentable subject matter under 35 USC §101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled//implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof, limited to patentable subject matter under 35U.S.C. §101. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, limited to patentable subject matter under 35 U.S.C. §101, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc) and so forth).

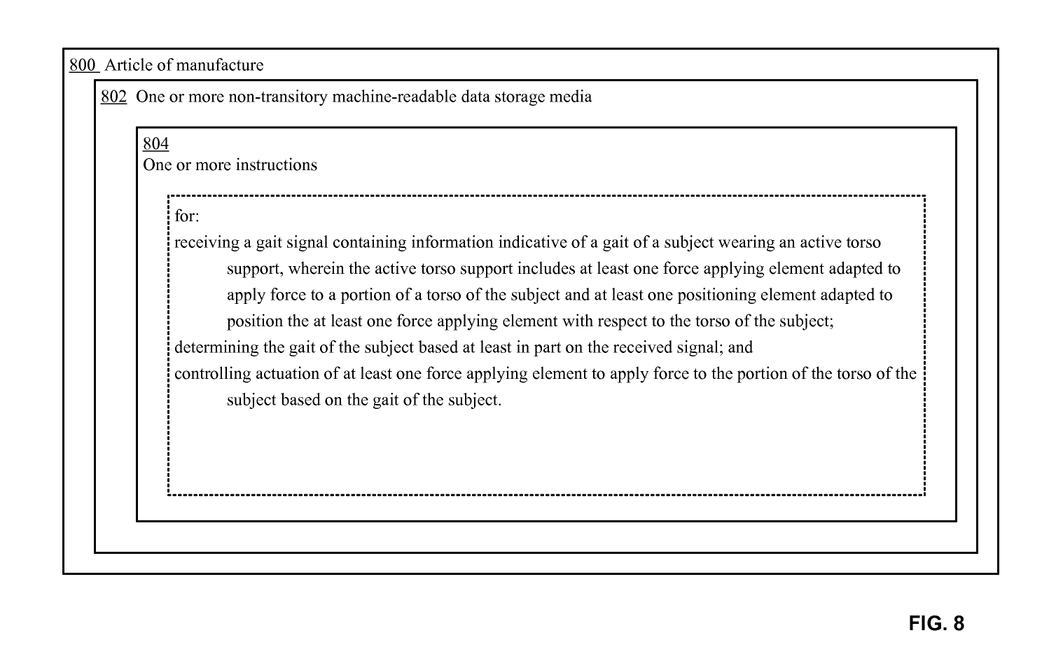

FIG. 8 depicts an article of manufacture 800 that includes one or more non-transitory machine-readable data storage media 802 bearing one or more instructions 804 for: receiving a gait signal containing information indicative of a gait of a subject wearing an active torso support, wherein the active torso support includes at least one force applying element adapted to apply force to a portion of a torso of the subject and at least one positioning element adapted to position the at least one force applying element with respect to the torso of the subject; determining the gait of the subject based at least in part on the received signal; and controlling actuation of at least one force applying element to apply force to the portion of the torso of the subject based on the gait of the subject.

Instructions 804 depicted in FIG. 8 correspond to method 400 shown in FIG. 4. Other variants of methods as depicted in FIGS. 4-7 and as described herein can be implemented through the use of non-transitory machine-readable data storage media bearing one or more suitable instructions.

In an aspect, the one or more non-transitory machine readable data storage media 802 bear one or more instructions 804 for receiving the gait signal from at least one sensor on the torso support. In another aspect, the one or more non-transitory machine readable data storage media 802 bear one or more instructions 804 for receiving the gait signal from a remote device.

The one or more non-transitory machine readable data storage media 802 may bear one or more instructions 804 for sensing neural activity in the subject, or one or more instructions 804 for sensing muscle activity in the subject.

In an aspect, the one or more non-transitory machine readable data storage media 802 bear one or more instructions 804 for controlling actuation of the at least one force applying element responsive to a change in the gait of the subject, for example, by controlling actuation of the at least one force applying element as a function of the speed of the gait.

In an aspect, the one or more instructions 804 for controlling actuation of at least one force applying element to apply force to the portion of the torso of the subject based on the gait of the subject include one or more instructions 804 for controlling actuation of the at least one force applying element to provide additional support to the torso of the subject responsive to detection of the change in the gait of the subject, or alternatively, to provide less support to the torso of the subject responsive to detection of the change in the gait of the subject.

In an aspect, the one or more instructions 804 for controlling actuation of at least one force applying element to apply force to the portion of the torso of the subject based on the gait of the subject include one or more instructions 804 for controlling actuation of the at least one force applying element responsive to change in the gait of the subject to walking, running, climbing stairs, descending stairs, or standing still.

In an aspect, the one or more instructions 804 for controlling actuation of at least one force applying element to apply force to the portion of the torso of the subject based on the gait of the subject include one or more instructions 804 for controlling actuation of the at least one force applying element responsive to change in the gait of the subject indicative of the subject stumbling, falling, or changing direction.

In an aspect, the one or more non-transitory machine readable data storage media 802 bear one or more instructions 804 for sensing an inclination signal indicative of an inclination of at least a portion of the active torso support. In connection therewith, the one or more non-transitory machine readable data storage media 802 may bear one or more instructions 804 for determining the gait of the subject based at least in part on the inclination signal. In addition, the one or more instructions 804 for controlling actuation of at least one force applying element to apply force to the portion of the torso of the subject based on the gait of the subject include one or more instructions 804 for controlling actuation of the at least one force applying element according to a temporal pattern based at least in part on the gait of the subject. The one or more non-transitory machine readable data storage media 802 may bear one or more instructions 804 for controlling actuation of at least two force applying elements on the active torso support configured to apply force to at least two different portions of the torso of the subject according to a spatial and temporal pattern based at least in part on the gait of the subject.

The one or more non-transitory machine readable data storage media may bear one or more instructions 804 for controlling actuation of the at least one force applying element according to a pre-defined pattern selected from a plurality of pre-defined patterns, based at least in part on the user input and/or upon the gait of the subject. The one or more non-transitory machine readable data storage media 802 may bear one or more instructions 804 for selecting the pre-defined pattern from a plurality of pre-defined patterns corresponding to a plurality of pre-defined gaits of the subject, for example, walking, running, climbing stairs, descending stairs, climbing a slope, descending a slope, and standing still.

In an aspect, the one or more instructions 804 for controlling actuation of at least one force applying element to apply force to the portion of the torso of the subject based on the gait of the subject include one or more instructions 804 for controlling a pattern of force generated by the at least one force generating element. In an aspect, the one or more instructions 804 for controlling actuation of at least one force applying element to apply force to the portion of the torso of the subject based on the gait of the subject include one or more instructions 804 for controlling a pattern of motion generated by the at least one force generating element.

The one or more non-transitory machine readable data storage media 802 bear one or more instructions 804 for delivering a thermal stimulus to a least a portion of the torso of the subject, delivering a stimulus to a neural structure in the torso of the subject, or delivering a stimulus to a muscle in the torso of the subject.

In an aspect, the one or more non-transitory machine readable data storage media 802 bear one or more instructions 804 for controlling actuation of the at least one force applying element to apply force to the torso of the subject, wherein at least a component of the force is in a direction normal to the surface of the torso of the subject. In an aspect, the one or more non-transitory machine readable data storage media 802 bear one or more instructions 804 for controlling actuation of the at least one force applying element to apply force to the torso of the subject, wherein at least a component of the force is in a direction tangential to the surface of the torso of the subject.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An active torso support, comprising:
at least one force applying element adapted to apply force to a portion of a torso of a subject;
at least one positioning element adapted to position the at least one force applying element with respect to the torso of the subject; and
control circuitry including:
gait analysis circuitry configured to determine a gait of the subject responsive to a signal containing information indicative of the gait of the subject; and
actuation circuitry configured to control actuation of the at least one force applying element responsive to the signal indicative of the gait of the subject.

2. The active torso support of claim 1, comprising:
at least one sensor carried by the at least one positioning element adapted to produce the signal containing information indicative of the gait of the subject.

3.-19. (canceled)

20. The active torso support of claim 1, wherein the active torso support is operably coupleable to a remote device located in the environment of the subject, the remote device including at least one sensor adapted to produce the signal containing information indicative of the gait of the subject.

21. The active torso support of claim 1, wherein the active torso support is operably coupleable to a remote device located on the body of the subject, the remote device including at least one sensor adapted to produce the signal containing information indicative of the gait of the subject.

22.-23. (canceled)

24. The active torso support of claim 1, wherein the actuation circuitry is configured to control actuation of the at least one force applying element responsive to a change in the gait of the subject.

25.-39. (canceled)

40. The active torso support of claim 1, including at least two force applying elements adapted to apply force to at least two different portions of the torso of the subject, wherein the actuation circuitry is configured to control actuation of the at least two force applying elements according to a spatial and/or temporal pattern based at least in part on the signal indicative of the gait of the subject.

41.-54. (canceled)

55. A method of controlling an active torso support, comprising:
receiving a gait signal containing information indicative of a gait of a subject wearing the active torso support, wherein the active torso support includes at least one force applying element adapted to apply force to a portion of a torso of the subject and at least one positioning element adapted to position the at least one force applying element with respect to the torso of the subject;
determining the gait of the subject based at least in part on the received signal; and
controlling actuation of at least one force applying element to apply force to the portion of the torso of the subject based on the gait of the subject.

56. The method of claim 55, including receiving the gait signal from at least one sensor on the torso support.

57.-61. (canceled)

62. The method of claim 55, including receiving the gait signal from a remote device.

63.-78. (canceled)

79. The method of claim 55, including controlling actuation of the at least one force applying element according to a temporal pattern based at least in part on the gait of the subject.

80. The method of claim 55, controlling actuation of at least two force applying elements on the active torso support configured to apply force to at least two different portions of the torso of the subject, by controlling actuation of the at least two force applying elements according to a spatial and temporal pattern based at least in part on the gait of the subject.

81. The method of claim 55, including controlling actuation of the at least one force applying element according to a pre-defined pattern selected from a plurality of pre-defined patterns.

82.-92. (canceled)

93. An article of manufacture comprising:
one or more non-transitory machine-readable data storage media bearing one or more instructions for:
receiving a gait signal containing information indicative of a gait of a subject wearing an active torso support, wherein the active torso support includes at least one force applying element adapted to apply force to a portion of a torso of the subject and at least one positioning element adapted to position the at least one force applying element with respect to the torso of the subject;
determining the gait of the subject based at least in part on the received signal; and
controlling actuation of at least one force applying element to apply force to the portion of the torso of the subject based on the gait of the subject.

94. The article of manufacture of claim 93, wherein the one or more non-transitory machine readable data storage media bear one or more instructions for receiving the gait signal from at least one sensor on the torso support.

95.-97. (canceled)

98. The article of manufacture of claim 93, wherein the one or more non-transitory machine readable data storage media bear one or more instructions for controlling actuation of the at least one force applying element responsive to a change in the gait of the subject.

99.-112. (canceled)

113. The article of manufacture of claim 93, wherein the one or more non-transitory machine readable data storage media bear one or more instructions for controlling actuation of at least two force applying elements on the active torso support configured to apply force to at least two different portions of the torso of the subject according to a spatial and temporal pattern based at least in part on the gait of the subject.

114. The article of manufacture of claim 93, wherein the one or more non-transitory machine readable data storage media bear one or more instructions for controlling actuation of the at least one force applying element according to a pre-defined pattern selected from a plurality of pre-defined patterns.

115.-126. (canceled)

127. The active torso support of claim 2, wherein the at least one sensor includes at least one of an accelerometer, a tri-axial accelerometer, an integrating accelerometer, a gyro sensor, a magnetometer, and a differential position sensor.

128. The active torso support of claim 1, comprising at least one of a sensor adapted to sense neural activity and a sensor adapted to sense muscle activity.

129. The active torso support of claim 24, wherein the actuation circuitry is configured to control actuation of the at least one force applying element to provide additional support to the torso of the subject responsive to detection of the change in the gait of the subject or provide less support to the torso of the subject responsive to detection of the change in the gait of the subject

130. The active torso support of claim 24, wherein the gait analysis circuitry is configured to detect a change in the gait of the subject to at least one of walking, running, climbing stairs, descending stairs, standing still, stumbling, subject falling, and changing direction.

131. The active torso support of claim 1, comprising an inclinometer adapted to generate an inclination signal indicative of an inclination of at least a portion of the active torso support, wherein the gait analysis circuitry is configured to generate a signal indicative of the gait of the subject based at least in part on the inclination signal, and wherein the actuation circuitry is configured to control actuation of the at least one force applying element responsive to the signal indicative of the gait of the subject based at least in part on the inclination signal.

132. The active torso support of claim 1, further comprising a memory adapted to store a plurality of pre-defined patterns, wherein the actuation circuitry is configured to control actuation of the at least one force applying element according to a pre-defined pattern selectable from the plurality of pre-defined patterns based on at least one of an input provided by a user via a user input and the gait of the subject.

133. The active torso support of claim 1, comprising at least one of a thermal stimulus source configured to deliver a thermal stimulus to a least a portion of the torso of the subject, a neural stimulus source configured to deliver a stimulus to a neural structure in the torso of the subject, and a muscle stimulator configured to deliver a stimulus to a muscle in the torso of the subject.

134. The method of claim 56, wherein receiving the gait signal includes receiving a signal from at least one of an accelerometer, a tri-axial accelerometer, a gyro, a magnetometer, and a differential position sensor.

135. The method of claim 55, including sensing at least one of neural activity in the subject and muscle activity in the subject.

136. The method of claim 55, including controlling actuation of the at least one force applying element to change the amount of support provided to the torso of the subject responsive to a change in the gait of the subject.

137. The method of claim 55, wherein controlling actuation of the at least one force applying element includes controlling actuation of the at least one force applying element responsive to a change in the gait of the subject indicative of at least one of one of walking, running, climbing stairs, descending stairs, standing still, stumbling, falling, and changing direction.

138. The method of claim 55, further comprising sensing an inclination signal indicative of an inclination of at least a portion of the active torso support and determining the gait of the subject based at least in part on the inclination signal.

139. The method of claim 81, comprising selecting the pre-defined pattern from the plurality of pre-defined patterns based at least in part on an input received from a user on a user input and based at least in part upon the gait of the subject.

140. The method of claim 55, wherein controlling actuation of the at least one force applying element includes controlling at least one of a pattern of force and a pattern of motion generated by the at least one force generating element.

141. The method of claim 55, including at least one of delivering a thermal stimulus to a least a portion of the torso of the subject, delivering a stimulus to a neural structure in the torso of the subject, and delivering a stimulus to a muscle in the torso of the subject.

142. The article of manufacture of claim 98, wherein the one or more instructions for controlling actuation of at least one force applying element to apply force to the portion of the torso of the subject based on the gait of the subject include one or more instructions for controlling actuation of the at least one force applying element responsive to change in the gait of the subject to one of walking, running, climbing stairs, descending stairs, standing still, stumbling, falling, and changing direction.

143. The article of manufacture of claim 93, wherein the one or more non-transitory machine readable data storage media bear one or more instructions for sensing an inclination signal indicative of an inclination of at least a portion of the active torso support and one or more instructions for determining the gait of the subject based at least in part on the inclination signal.

144. The article of manufacture of claim 114, wherein the one or more non-transitory machine readable data storage media bear at least one of one or more instructions for receiving a user input from a user and selecting the pre-defined pattern from the plurality of pre-defined patterns based at least in part on the user input and one or more instructions for selecting the pre-defined pattern from the plurality of pre-defined patterns based at least in part upon the gait of the subject.
